SEQUENCE LISTING (2 sequences)

SEQ ID NO: 1 - protein, 8 aa, synthetic construct
DYKDDDDK

SEQ ID NO: 2 - DNA, 30 bp, synthetic construct
  source 1..30: mol_type = other DNA
aaaaaaaaaaaatgctcgggaaggctactc

(19) United States
(12) Patent Application Publication
(10) Pub. No.: US 2025/0003959 A1
(43) Pub. Date: Jan. 2, 2025

(54) CELL ANALYSIS METHODS, COMPOSITIONS, AND USES

(71) Applicants: Institute for Systems Biology, Seattle, WA (US); The Regents of the University of California, Riverside, CA (US)

(72) Inventors: Min Xue, Chino Hills, CA (US); Wei Wei, Seattle, WA (US); Zhili Guo, Riverside, CA (US); Hanjun Cheng, Seattle, WA (US); Zhonghan Li, Riverside, CA (US)

(73) Assignees: Institute for Systems Biology, Seattle, WA (US); The Regents of the University of California, Riverside, CA (US)

(21) Appl. No.: 18/576,905

(22) PCT Filed: Jul. 1, 2022
(86) PCT No.: PCT/US2022/073378

§ 371 Date: Jan. 5, 2024

Related U.S. Application Data
(60) Provisional application No. 63/219,272, filed on Jul. 7, 2021.

(51) Int. Cl. G01N 33/543; G01N 33/542

(52) U.S. Cl. CPC G01N 33/54353; G01N 33/542; G01N 2500/10

(57) ABSTRACT

A chemical approach is provided to profile uptake of compounds in cells, alone or in combination with other analytical techniques. The disclosure is particularly well suited for detecting cellular uptake of metabolites such as fatty acids. The disclosure can be carried out, applied in solution or on a surface, in bulk or on single cells, and is compatible with one or more additional techniques such as protein analysis. The disclosure is exemplified by probing fatty acid influx alone and in combination with proteomics analysis on a single-cell barcode chip to identify a combination therapy for inhibiting fatty acid metabolism and treating cancer.

### CROSS REFERENCE TO RELATED APPLICATIONS

This application claims the benefit of U.S. Provisional Patent Application No. 63/219,272, filed Jul. 7, 2021, which is incorporated here by reference in its entirety.

### ACKNOWLEDGMENT OF GOVERNMENT SUPPORT

This invention was made with government support under grant numbers R03 CA227352, U54 CA199090, and U01 CA217655 awarded by the National Institutes of Health. The government has certain rights in the invention.

### FIELD

This disclosure relates to methods and compositions related to cell analysis.

### BACKGROUND

Similar to glucose and amino acids, fatty acids are a major energy source required for sustaining cellular growth and proliferation (Currie et al. (2013) Cell Metab 18 (2): 153-61). Abnormal fatty acid metabolism is frequently observed in cancer, but its regulatory mechanisms and therapeutic implications are unclear. Compared with the well-studied glucose and amino acid metabolic patterns in cancer cells (Pavlova et al. (2016) Cell Metab 23 (1): 27-47; and Martinez-Outschoorn et al. (2017) Nat Rev Clin Oncol 14 (1): 11-31), altered fatty acid metabolism received less attention, and current research primarily focuses on the de novo fatty acid synthesis pathway (Carracedo et al. (2013) Nat Rev Cancer 13 (4): 227-32). However, increasing evidence demonstrates that cancer cells' survival and metastatic spreading often require exogenous fatty acid uptake and consumption, even in cells exhibiting high lipogenic activities (Kamphorst et al. (2013) Proc Natl Acad Sci USA 110 (22): 8882-7; Raynor et al. (2015) Lipids Health Dis 14:69; Beloribi-Djefaflia et al. (2016) Oncogenesis 5: e189; Liu et al. (2010) Anticancer Res 30 (2): 369-74; Bensaad et al. (2014) Cell Rep 9 (1): 349-65; and Camarda et al. (2016) Nat Med 22 (4): 427-32). In addition, fatty acid metabolism in tumor cells exhibits significant plasticity-even the same set of cells can alter their fatty acid dependence in response to drug or environmental stress (Bensaad et al. (2014) Cell Rep 9 (1): 349-65; and Du et al. (2020) Nat Commun 11 (1): 4830-4830). These studies high-light the relevance and complexity of fatty acid metabolism in cancer and necessitate more comprehensive research.

The study of cancer cell metabolism can be confounded by prominent cellular heterogeneity. The causes of this heterogeneity include genetic diversity, signaling and metabolic pathway redundancy, and local microenvironment variations. Such heterogeneity enables metabolic flexibility and promotes tumor progression. On the other hand, these metabolic abnormalities also render tumor cells more vulnerable to metabolic perturbations, which may be exploited therapeutically (Bensaad et al. (2014) Cell Rep 9 (1): 349-65; Tateishi et al. (2016) Clin Cancer Res 22 (17): 4452-65; and Hensley et al. (2016) Cell 164 (4): 681-694). Therefore, deciphering cancer cell metabolism, especially in the context of oncogenic signaling heterogeneity, promises the rational design of combinatory therapies for synergistic intervention. Such a task calls for a multiplex single-cell analytical assay that can simultaneously profile metabolic activity and protein levels.

Current solutions for detecting fatty acid uptake in cells include fluorophore-modified fatty acid analogs and isotopically labeled fatty acid analogs (e.g., Huang et al. (2002) J Biol Chem 277 (32): 29139-5; and Witney et al. (2014) J Nucl Med 55 (9): 1506-12). Unfortunately, working with isotopically labeled fatty acid analogs is expensive and requires special facilities, handling, disposal, and training. Fluorophore-modified fatty acids suffer from reliance on large modification groups that compromise the analogs' abilities to mimic natural fatty acids. Expensive and specialized instruments are also necessary to perform the analysis. None of the methods are easily compatible with protein analysis.

The above drawbacks make fatty acid analyses especially problematic for multiomics, and particularly for single-cell analytics. For example, there is no method that can detect fatty acid uptake from single cells while being compatible with protein analysis. There are also no surface-based methods that can detect fatty acid uptake from single cells.

There is a need for methods and compositions to detect the uptake of compounds such as metabolites in cells, and particularly for detecting fatty acid uptake compatible with single-cell and protein analysis. The present disclosure addresses these and other needs.

### SUMMARY

Methods, compositions, and devices (such as surfaces) are provided to profile the uptake of compounds in a cell. Each are compatible with analysis of other compounds and molecules. The disclosure is exemplified by measuring fatty acid influx alone and together with proteomics analysis on a single-cell barcode chip to identify a combination therapy for inhibiting fatty acid metabolism and treating cancer.

Provided herein are surfaces including a dendrimer terminating in a plurality of densely packed azide-capturing groups, the dendrimer conjugated to a spacer including a single stranded nucleic acid hybridized to a complementary nucleic acid patterned on the surface in a spatially addressable array. In some examples, the spacer and the complementary nucleic acid include complementary barcode nucleic acids patterned on the surface in a spatially addressable barcoded array. In some examples, the surface is on a single-cell barcode chip. In some examples, the spacer is labeled with a reporter dye. In some examples, the surface further includes a capture antibody reagent conjugated to a second spacer including a barcode patterned on the surface in a spatially addressable barcoded array. In further examples, the dendrimer and the capture antibody reagent are each individually spatially patterned in stripes in a microchamber of a single-cell barcode chip. In some examples, the azide-capturing groups are dibenzocyclooctyne (DBCO). For example, the dendrimer is selected from G-3 DBCO dendrimer, G-4 DBCO dendrimer, and derivatives thereof.

Also provided are cell-free compositions including a purified, monodisperse dendrimer terminated with a plurality of densely packed azide-capturing groups, the dendrimer conjugated to a spacer. In some examples, the spacer includes a single stranded nucleic acid, for example, a barcode nucleic acid. In some examples, the spacer includes a reporter dye. For example, the reporter dye is a fluorophore, such as Cy3. In some examples, the azide-capturing groups are dibenzocyclooctyne (DBCO). For example, the dendrimer is selected from G-3 DBCO dendrimer, G-4 DBCO dendrimer, and derivatives thereof.

Further provided are compositions including an azide-modified quencher of a reporter dye, the azide-modified quencher including a hydrophilic polymer bearing an azide moiety and a quencher of the reporter dye. In some examples, the quencher of a reporter dye is a dark quencher. In additional examples, the hydrophilic polymer includes monomers selected from hydrophilic amino acid, polyethylene glycol, and combinations thereof. In some examples, the azide moiety is azido lysine. In further examples, the dark quencher is a Black Hole Quencher® selected from BHQ®-1, BHQ®-2, and BHQ®-3, such as BHQ2-N₃ and derivatives thereof.

Also provided herein are assays that include contacting a disclosed surface with an azide-modified compound of interest and an azide-modified detection reagent under competitive binding conditions and detecting the azide-modified detection reagent. In some examples, the azide-modified compound of interest is an azide-modified metabolite, such as an azide-modified fatty acid, for example, azidopentanoic acid. In some examples, the spacer is labeled with a fluorophore and the azide-modified detection reagent includes a quencher of the fluorophore, and the detecting includes measuring fluorescence resonance energy transfer (FRET). In some examples, the azide-modified quencher is selected from BHQ2-N₃ and derivatives thereof. In other examples, the azide-modified detection reagent is an azide-Flag tag. In additional examples, detecting the azide-Flag tag includes contacting the surface with a fluorophore-labeled anti-Flag tag antibody and measuring fluorescence thereof. In some examples, the assay further includes a capture antibody reagent, and the method further includes contacting the surface with a protein sample and a detection antibody reagent capable of binding a protein of interest, and detecting binding of the detection antibody reagent to the protein of interest if present in the protein sample. In some examples, the detecting assesses the presence or absence of the protein of interest in the protein sample, and optionally, the level of the protein of interest in the protein sample. In some examples, the detection antibody reagent is a fluorophore-labeled anti-protein of interest antibody, and the detecting includes measuring fluorescence thereof. In some examples, the capture antibody reagent binds a protein of interest selected from the group consisting of phospho-p70 S6 kinase, EGFR, phospho-ERK1, NDRG1, phospho-Src, 4EBP1, phospho-Akt, Ki-67/MKI6, mutant proteins thereof, and combinations thereof. In further examples, the azide-modified compound of interest is a metabolite, and the metabolite and the protein of interest are detected in an isolated region on the same surface, such as a microchamber of a single-cell barcode chip. In some examples, the detecting is on a single cell.

In some examples, the assay further includes contacting a cell with the azide-modified compound of interest and lysing the cell so as to release the azide-modified compound of interest taken up by the cell. In some examples, the contacting and lysing steps are prior to contacting the surface with the azide-modified compound of compound of interest and the azide-modified detection reagent. In some examples, the cell is a cancer cell. In other examples, the cell is a single cell in isolation.

Further provided herein are methods of treating a cancer patient, the method including administering to a cancer patient in need thereof an effective amount of an inhibitor of fatty acid metabolism in combination with an effective amount of an inhibitor of a protein selected from p70S6K, pEGFR, or combinations thereof. In some examples the inhibitors of fatty acid metabolism, p70S6K, and pEGFR, are trimetazidine, LY2584702, and erlotinib, respectively. In other examples, the cancer is selected from glioblastoma and colon cancer.

The disclosure provides several advantages over the prior art. The methods, compositions, and devices are particularly well suited for measuring uptake of metabolites such as fatty acids that are minimally chemically perturbed by addition of an azide group. The methods, compositions, and devices are more powerful as they are widely compatible with multiple systems, for example, they can be carried out in solution or on a surface, in bulk or with single cells, and multiplexed with a variety of multi-omics techniques such as protein analysis. Another advantage is that the dendrimers and detection system capture and produce a signal sufficient for measuring in a concentration-dependent manner the uptake of azide-modified fatty acid analogs at physiologically relevant concentrations suitable for single-cell analysis. The methods, compositions, and devices of the disclosure are also less expensive because complicated instruments such as a positron emission tomography machines are not required. Another advantage is versatility in that it can be adapted to detect any azide containing small molecules alone or in combination with the detection of other compounds and/or molecules. Thus, the methods, compositions, and devices find use beyond azide-modified fatty acids including detection of any azide containing compounds, particularly in combination with protein analysis, and more particularly at a single-cell level.

The foregoing and other features of the disclosure will become more apparent from the following detailed description, which proceeds with reference to the accompanying figures.

### DESCRIPTION OF THE FIGURES

FIGS. 1A and 1B depict the detecting of fatty acid from single cells. FIG. 1A: Cells take up azide-modified fatty acid (FA-N3) molecules and subsequently release them upon lysis. FIG. 1B: Cy3-modified dibenzocyclooctyne (DBCO) dendrimers capture the released FA-N3 molecules. The FA-N3 competes with the BHQ2-N3 quencher to retain the Cy3 fluorescence.

FIG. 2A depicts synthesis of the G-3 DBCO dendrimer conjugated with Cy3-ssDNA. The amine-bearing G-3 dendritic scaffold was constructed through solid-phase peptide synthesis (SPPS) and then cleaved off from the resin. The scaffold was later conjugated with Cy3-modified ssDNA via SPAAC. Finally, all of the primary amines on the dendrimer scaffold were capped using DBCO-sulfo-NHS ester. G-4 DBCO dendrimer was prepared in a similar manner with an additional round of lysine conjugation.

FIG. 2B depicts synthesis of BHQ2-N3. Lysine and polyethylene glycol residues were necessary to increase the aqueous solubility.

FIGS. 3A-3G depict: FIG. 3A: A picture of the mini well assay device. The PDMS slab provides assay space, and the glass slide enables surface-based detection. The dendrimer-ssDNA conjugate can be immobilized through DNA hybridization. The resulting surface fluorescence can be quantified using a microarray scanner. FIG. 3B: BHQ2-N3 effectively reacted with the DBCO dendrimer and quenched the Cy3 fluorescence. Both G-3 and G-4 dendrimers exhibited quenching. FIG. 3C: Different concentrations of BHQ2-N3 competing with 100 μM of FA-N₃ for the DBCO dendrimer binding sites. The attachment of BHQ2-N₃ caused quenching of the Cy3 fluorescence. FIG. 3D: The fluorescence response curve generated by varying the concentrations of the FA-N₃ probe. FIG. 3E: FA-N₃ (up to 20 mM) was well tolerated by U87VIII cells. FIG. 3F: Incubating U87VIII cells with varying concentrations of FA-N3 led to different fluorescence intensities on the dendrimer-based detection platform. FIG. 3G: Low temperature (4° C.) incubation inhibited the FA-N₃ uptake, and fixed cells did not exhibit uptake either. The cell lysate was used to provide background fluorescence intensity. The error bars in all graphs (FIGS. 3B-3G) show the standard deviation values calculated from four individual measurements.

FIGS. 4A-4E depict: FIG. 4A: A picture of the assembled SCBC device. The PDMS microfluidic device contains microchambers that are controlled by the pneumatic valves. The circle highlights the trapped single U87VIII cell in this example. The glass slides at the bottom contain pre-patterned ssDNA barcode stripes, which enables multiplex immunofluorescence-based protein detection. After the assay, the barcode fluorescence intensities are quantified using a microarray scanner, and the values are extracted and assigned according to the chamber location. A representative fluorescence image of the barcode strips from one single-cell chamber is shown here as an example. FIG. 4B: The fluorescence response curve generated using different concentrations of the FA-N₃ probe. The error bars show the standard deviation values calculated from four individual measurements. The data points were fitted using a Hill function. FIG. 4C: The multiplex single-cell dataset obtained from U87VIII cells. Each dot shows an analyte level obtained from a single cell. The boxes depict the middle two quartiles of the analyte level distributions, and the internal dots represent the median values. A total of 151 single cells were assayed using two SCBC devices. FIG. 4D: The measured fatty acid amount from each single cell plotted against the diameter of the cell. No obvious correlation existed between these two parameters. FIG. 4E: The violin plots represent the standardized analyte level distributions. The dataset was standardized for each analyte to obtain the Z score of each value. The widths of the violin plots represent the observed frequencies.

FIGS. 5A-5D depict: FIG. 5A: Agglomerative hierarchical clustering (AHC) of the single-cell dataset. The standardized single-cell dataset was used as the input. The Euclidean distance values between data points were calculated and tabulated as a matrix. This distance matrix was then used to perform agglomerative hierarchical clustering using Ward's method. Each small bar represents one single-cell data point, and the color represents the Z score of that data point. This AHC analysis identified two distinct clusters, which are denoted by the top and bottom branches in the dendrogram on the left. FIG. 5B: Analyte levels of the two cluster centroids. A distinct bifurcation was observed for most analytes. FIG. 5C: The loading plot showing the first two principal components (PC1 and PC2) of the single-cell dataset. The vectors are labeled with the corresponding analyte names, and the position of the vectors show the loading of the analytes in each PC. FIG. 5D: The correlation network of the analytes. The Spearman correlation values were calculated between each analyte pair. All lines represent negative correlations, except for the two labeled “+”. The line thickness corresponds to the correlation level.

FIGS. 6A-6C depict: FIG. 6A: Illustration of the experimental procedure. FIG. 6B: U87VIII cell viabilities as results of LY2584702 (p70S6K inhibitor, 10 μM) and trimetazidine (fatty acid metabolism inhibitor, 1 μM) treatments. The error bars show the standard deviation values calculated from three individual samples. FIG. 6C: Synergy scores calculated from the BLISS method across different concentrations of trimetazidine and LY2584702 combinations.

FIGS. 7A and 7B depict: FIG. 7A: Co-administering trimetazidine and erlotinib results in synergistic effects across different drug concentrations. FIG. 7B: Synergistic killing of U87VIII cells using a combination of 1 μM of trimetazidine and 1 μM of erlotinib.

FIGS. 8A and 8B depict FIG. 8A: azide-modified fatty acid analog competes with azido-Flag (azide-DYKDDDDK; SEQ ID NO: 1) for the binding sites of the dendrimers. These Flag tags are detected by anti-Flag antibodies modified with AF647. FIG. 8B: Results of competitive binding with azido-Flag and detection with AF647. The surface fluorescence intensities negatively correlate with the FA-N3 concentrations.

### SEQUENCE LISTING

Any nucleic acid and amino acid sequences listed herein or in the accompanying sequence listing are shown using standard letter abbreviations for nucleotide bases and amino acids, as defined in 37 C.F.R. § 1.822. In at least some cases, only one strand of each nucleic acid sequence is shown, but the complementary strand is understood as included by any reference to the displayed strand.

SEQ ID NO: 1 is the amino acid sequence of a Flag tag.

SEQ ID NO: 2 is the nucleic acid sequence of a Cy3-labeled oligonucleotide.

### DETAILED DESCRIPTION

As described in greater detail below, a chemical approach is provided to profile compound uptake alone or in combination with protein profiling in cells. Profiling uptake, particularly metabolite uptake in cells, and more specifically, fatty acid uptake in cells alone or in combination with protein analysis is accomplished by application of (i) azide-modified analogs to probe compound influx, and (ii) surface-immobilized dendrimers with a plurality of densely packed azide-capturing groups for detection. Integration in a microfluidic platform provides streamlined isolation and functional analysis of target cells from complex biological samples in single-cell format.

### Methods of Detection

Provided herein are assays that include contacting a disclosed surface with an azide-modified compound of interest and an azide-modified detection reagent under competitive binding conditions and detecting the azide-modified detection reagent. In some examples, the methods generally involve detecting influx of a compound into a cell. In one example, the method includes contacting a cell with an azide-modified compound of interest, lysing the cell so as to release the azide-modified compound of interest taken up by the cell, capturing the released azide-modified compound of interest with a dendrimer terminated in a plurality of densely packed azide-capturing groups, and detecting the azide-modified compound of interest captured by the dendrimer. In some examples, the azide-modified compound of interest is an azide-modified metabolite. A specific example of an azide-modified metabolite is an azide-modified fatty acid. Azidopentanoic acid is a specific example of an azide-modified metabolite.

In some examples, detection is carried out by contacting the dendrimer with an azide-modified detection reagent and measuring binding of the reagent to the dendrimer. In this example, the capturing step and contacting of the dendrimer with the azide-modified detection reagent can be carried out in various arrangements suitable for a given end use, such as a competitive assay, and more specifically, such as achieved by a surface-based competitive assay. For example, in many examples, the contacting of the dendrimer with the azide-modified detection reagent is carried out after the capturing. In other examples, the contacting of the dendrimer with the azide-modified detection reagent is in combination with the capturing step. In general, the contacting of the dendrimer with the azide-modified detection reagent is under conditions so that the amount of azide-modified compound of interest captured by the dendrimer correlates with binding of the azide-modified detection reagent in a concentration-dependent manner.

The azide-modified detection reagent is selected depending on the detection method of interest. For example, in certain examples, the azide-modified detection reagent is selected from (i) an azide-modified quencher of a reporter dye, such as for a competitive FRET detection, (ii) an azide-modified peptide, such as for competitive antibody detection, or (iii) a combination of (i) and (ii). When the azide-modified detection reagent is an azide-modified quencher of a reporter dye, the reporter dye is in some examples conjugated to the dendrimer, as described in greater detail below, and detection may be by measuring fluorescence resonance energy transfer (FRET). When the azide-modified detection reagent is an azide-modified peptide, the azide-modified peptide provides a binding site for a detection antibody or antibody detection system. An example of the azide-modified peptide is an azide-containing FLAG-tag reagent, which can be specifically bound by an anti-FLAG-tag detection antibody, or antibody capture and antibody detection system.

In some examples, the lysing step is in single-cell isolation so as to release contents of the cell into a region separated from other cells. An example is a single-cell microfluidic device such as a single-cell barcode chip system capable of retaining the cell in a region suitable for single-cell analysis. In some examples, the dendrimer is immobilized on a surface through a spacer, and the spacer comprises a single stranded nucleic acid hybridized to a complementary nucleic acid immobilized on the surface. In some examples, the single stranded nucleic acid, the complementary nucleic acid, or a combination thereof is barcoded.

A particular aspect is where the azide-modified compound of interest is a metabolite, and the surface further includes a capture antibody reagent and the method further includes contacting the surface with a protein sample and a detection antibody reagent capable of binding a protein of interest, and detecting binding of the detection antibody reagent to the protein of interest, if present in the protein sample. In some examples, the method assesses the presence or absence of the protein of interest in the protein sample, and may also asses the level of the protein of interest in the protein sample. In some examples, the detection antibody reagent is a fluorophore-labeled anti-protein of interest antibody, and the detecting includes measuring fluorescence thereof. In some examples, the capture antibody reagent binds a protein of interest selected from the group consisting of phospho-p70 S6 kinase, EGFR, phospho-ERK1, NDRG1, phospho-Src, 4EBP1, phospho-Akt, Ki-67/MKI6, mutant proteins thereof, and combinations thereof. In further examples, the azide-modified compound of interest is a metabolite, and the metabolite and the protein of interest are detected in an isolated region on the same surface, such as a microchamber of a single-cell barcode chip. In some examples, the detecting is on a single cell.

In some examples, the capture antibody is immobilized on a surface through a spacer, and the spacer comprises a single stranded nucleic acid sequence hybridized to a complementary nucleotide sequence immobilized on the surface. In many examples, the dendrimer, the capture antibody, or each individually the dendrimer and the capture antibody, is immobilized in a spatially addressable array patterned on the surface with the complementary nucleic acid, and the surface is on a single-cell barcode chip, flow cell, slide, bead, well, plate, dish, or combinations thereof. In some examples, the method includes capturing with a capture antibody at least one protein of interest released by lysing the cell as described above, and detecting the protein of interest captured by the capture antibody.

A featured aspect of the method involves characterizing fatty acid metabolism and oncogenic signaling in a cancer cell. In one example the method includes detecting influx of a compound into a cell as described above, wherein the cell is a cancer cell, the azide-modified compound of interest is an azide-modified fatty acid, the protein of interest is an oncogenic signaling protein, and the method further comprises: characterizing fatty acid metabolism and oncogenic signaling in the cancer cell by assessing uptake of the azide-modified fatty acid and level of the oncogenic signaling protein in the cell. In some examples, the level of the oncogenic signaling protein is positively or negatively correlated with fatty acid metabolism, and the oncogenic signaling protein is negatively correlated with fatty acid metabolism. In some examples, the cancer cell is obtainable from a cancer patient, and the method further comprises: identifying one or more therapeutics that target a protein of the cancer cell characterized as being a potential regulator of fatty acid metabolism based on uptake of the azide-modified fatty acid and level of the oncogenic signaling protein in the cell; and treating the patient with an effective amount of the one or more therapeutics.

In another example, a single-cell method for multiplex proteomic and metabolomic analysis is provided. This method includes: (a) contacting a cell with an azide-modified metabolite; (b) lysing the cell in single-cell isolation so as to release in single-cell isolation (i) proteins of the cell, and (ii) the azide-modified metabolite taken up by the cell; (c) capturing one or more of the proteins and the azide-modified metabolite released by the lysing on a surface in the single-cell isolation, the one or more proteins captured with one or more capture antibodies immobilized as a barcoded array on the surface, the azide-modified metabolite captured with a dendrimer immobilized on the surface and terminated in a plurality of azide-capturing groups; and (d) detecting (i) the one or more proteins captured by the one or more capture antibodies with one or more detection antibodies, and (ii) the azide-modified metabolite captured by the dendrimer with an azide-modified detection reagent.

Also provided is a method of treating a cancer patient, the method comprising administering to a cancer patient in need thereof an effective amount of an inhibitor of fatty acid metabolism in combination with an effective amount of an inhibitor of a protein selected from pEGFR, p70S6K, or combinations thereof. In some examples, the inhibitors of fatty acid metabolism, p70S6K, and pEGFR, are trimetazidine, LY2584702, and erlotinib, respectively. In some examples, the treating is for a cancer selected from glioblastoma or colon cancer.

### Azide-Modified Dendrimers and Compositions

Provided herein are compositions including a dendrimer terminated with a plurality of densely packed azide-capturing groups, the dendrimer conjugated to a spacer. In some examples, the composition is a cell-free composition. In additional examples, the dendrimer is a purified, monodisperse dendrimer terminated with a plurality of densely packed azide-capturing groups conjugated to a spacer. In some examples, the spacer includes a nucleic acid, such as a single-stranded nucleic acid. The nucleic acid is in some examples a single stranded DNA sequence, and in some examples, includes a barcode nucleic acid. In certain examples, the spacer is labeled with a reporter dye, such as a fluorophore. Numerous types of fluorophores can be employed. An exemplary fluorophore is Cy3.

Various azide-capturing groups can be used, such as groups for copper-catalyzed azide-alkyne cycloaddition chemistry or copper-free chemistry reactions. A particular azide-capturing group of the dendrimer is dibenzocyclooctyne (DBCO), a “copper-free” cycloaddition alkyne reagent. In some examples, the dendrimer is selected from G-3 DBCO dendrimer, G-4 DBCO dendrimer, and derivatives thereof.

Compositions comprising the dendrimers immobilized on a surface through the spacer are provided. Examples of a surface of particular interest include a surface on a single-cell barcode chip, flow cell, slide, bead, well, plate, dish, or combinations thereof.

In some examples, provided herein are surfaces including a dendrimer terminating in a plurality of densely packed azide-capturing groups, the dendrimer conjugated to a spacer including a single stranded nucleic acid hybridized to a complementary nucleic acid patterned on the surface in a spatially addressable array. In some examples, the spacer and the complementary nucleic acid includes complementary barcode nucleic acids patterned on the surface in a spatially addressable barcoded array. In some examples, the surface is on a single-cell barcode chip. In some examples, the spacer is labeled with a reporter dye. In some examples, the surface further includes a capture antibody reagent conjugated to a second spacer including a barcode patterned on the surface in a spatially addressable barcoded array. In further examples, the dendrimer and the capture antibody reagent are each individually spatially patterned in stripes in a microchamber of a single-cell barcode chip. In some examples, the azide-capturing groups are dibenzocyclooctyne (DBCO). For example, the dendrimer is selected from G-3 DBCO dendrimer, G-4 DBCO dendrimer, and derivatives thereof.

In some examples, the dendrimer composition or surface including the dendrimer composition further includes a composition selected from: (i) an azide-modified compound of interest, such as a metabolite, for example, a fatty acid; (ii) an azide-modified detection reagent, such as an azide-modified quencher of the reporter dye described in greater detail below; or (iii) combinations thereof. For example, when the dendrimer composition is labeled with a reporter dye, the dendrimer composition will often include an azide-modified quencher of the reporter dye.

### Azide-Modified Quenchers

Additional compositions include an azide-modified quencher of a reporter dye. The azide-modified quencher includes a hydrophilic polymer bearing an azide moiety and a quencher of the reporter dye. Reporter dye-labeled dendrimer and azide-modified quencher pairs are also provided.

The azide-modified quencher of the reporter dye is generally of the formula X—Y—Z, Y—X—Z, or X—Z—Y, wherein X is a hydrophilic moiety, Y is an azide moiety, and Z is a quencher of the reporter dye.

In some examples, the hydrophilic polymer includes monomers selected from hydrophilic amino acid, polyethylene glycol, and combinations thereof. In some examples, the azide moiety is azido-lysine. In some examples, the quencher of a reporter dye is a dark quencher, such as a Black Hole Quencher®. Of particular interest are Black Hole Quenchers® selected from BHQ®-1, BHQ®-2, and BHQ®-3. An exemplary azide-modified quencher is selected from BHQ2-N₃ and derivatives thereof.

### Single-Cell Barcode Chips

Devices of the disclosure include a single-cell barcode chip. Of particular interest is a single-cell barcode chip comprising a dendrimer terminated with a plurality of densely packed azide-capturing groups, the dendrimer conjugated to a spacer comprising a single stranded nucleic acid such as a single stranded DNA, hybridized to a complementary nucleic acid immobilized or patterned on a surface of the single-cell barcode chip. In many examples, the spacer and the complementary nucleic acid comprise complementary barcode nucleic acids patterned on the surface in a spatially addressable barcoded array.

In some examples, the single-cell barcode chip further includes an antibody conjugated to a second spacer, such as a single stranded nucleic acid, such as a single stranded DNA, immobilized on a surface of the single-cell barcode chip. In as many examples, the dendrimer and the antibody are each individually immobilized on the same surface. In further examples, the dendrimer and the antibody are immobilized on the surface in a spatially addressable microarray format, such as patterned single-stranded DNA barcode stripes.

A single-cell barcode chip of specific interest comprises (i) a dendrimer terminated with a plurality of densely packed azide-capturing groups, and (ii) an antibody, wherein the dendrimer and the antibody are each individually conjugated to a spacer comprising a barcoded single stranded nucleic acid immobilized on a surface of the single-cell barcode chip in a spatially addressable microarray format.

### Kits

Also provided are kits comprising one or more compositions and/or devices of the disclosure.

### EXAMPLES

The following examples are provided to illustrate certain particular features and/or examples. These examples should not be construed to limit the disclosure to the particular features or examples described.

Development, synthesis, and characterization of the dendrimer probes and BHQ2-N3, operation of SCBC, cell culture, data analysis, and other experimental details are described in Examples 1 through 17. The results and discussion, including general approach and experimental findings, are described in Example 18.

### Example 1: Materials

Rink Amide MBHA resin (loading capacity 0.68 mmol/g) was purchased from Aapptec (Louisville, KY). All of the Fmoc-protected amino acids were purchased from Anaspec (Fremont, CA) except Fmoc-1-Lys (Fmoc)-OH and Fmoc-Lys (N₃)—OH (Az4), which were purchased from Chem-Impex (Wood Dale, IL). BHQ2 carboxyl acid and Fmoc-PEG5-OH were purchased from Biosearch Technologies (Petaluma, CA) and BroadPharm (San Diego, CA), respectively. S-HyNic and S-4FB were purchased from TriLink BioTechnologies (San Diego, CA). The coupling reagent 2-(1H-benzotriazol-1-yl)-1,1,3,3-tetramethyluronium hexafluorophosphate (HBTU, 99.6%) was obtained from Chem-Impex (Wood Dale, IL). Diisopropylethylamine (DIEA, 99.5%) was purchased from ACROS (Germany). Triisopropylsilane (TIPS) was obtained from TCI (Portland, OR). Piperidine was purchased from Alfa Aesar (Ward Hill, MA). α-cyano-4-hydroxycinnamic acid (CHCA) was obtained from Sigma-Aldrich (St. Louis, MO). Tris base, sodium phosphate dibasic anhydrous (Na₂HPO₄, 99.6%), sodium phosphate monobasic monohydrate (NaH₂PO₄, 99.4%), sodium chloride (NaCl), ascorbic acid, Tween 20, sodium dodecyl sulfate (SDS), bovine serum albumin (BSA), acetonitrile (CH₃CN), diethyl ether (Et₂O), ethyl acetate (EA), N,N′-dimethylformamide (DMF), and dichloromethane (DCM) were purchased from Thermo Fisher Scientific (Waltham, MA). DNA-Cy3-NH2 (5′-/5AmMC6/AAAAAAAAAAAA/iCy3/TGCTCGGGAAGGCTACTC-3′; SEQ ID NO: 2) was ordered from Integrated DNA Technologies, Inc. (Coralville, IA). DBCO-C6-Sulfo-NHS was purchased from Click Chemistry Tools (Scottsdale, AZ).

TABLE 1
Antibodies employed

| Capture antibody name | Manufacture |
|---|---|
| Phospho-p70 S6 Kinase (T389) DuoSet | R&D Systems, DYC896 |
| Human EGFR Antibody, Goat Polyclonal | R&D Systems, AF231 |
| Human/Mouse/Rat Phospho-ERK1 | R&D Systems, DYC1825 |
| (T202/Y204) DuoSet | |
| Human NDRG1, Goat Polyclonal | R&D Systems, AF5209 |
| Human Phospho-Src (Y419) DuoSet | R&D Systems, DYC2685 |
| Human/Mouse 4EBP1 antibody, Goat | R&D Systems, AF3227 |
| Polyclonal | |
| Human/Mouse Phospho-Akt1 (S473) DuoSet | R&D Systems, DYC2289C |
| Human Ki-67/MKI67, Sheep Polyclonal | R&D Systems, AF7617 |
| Detection antibody name | Manufacture |
| Phospho-p70 S6 Kinase (T389) DuoSet | R&D Systems, DYC896 |
| Phospho-EGF Receptor (Tyr1173) | Cell Signaling, 4407S |
| Antibody, Rabbit Monoclonal | |
| Human/Mouse/Rat Phospho-ERK1 | R&D Systems, DYC1825 |
| (T202/Y204) DuoSet | |
| Phospho-NDRG1 (Thr346) Antibody, | Cell Signaling, 7497S |
| Rabbit Monoclonal | |
| Human Phospho-Src (Y419) DuoSet | R&D Systems, DYC2685 |
| Phospho-4E-BP1 (Thr37/46) Antibody, | Cell Signaling, 5132S |
| Rabbit Monoclonal | |
| Human/Mouse Phospho-Akt1 (S473) DuoSet | R&D Systems, DYC2289C |
| Ki-67 Antibody, Rabbit Monoclonal | Cell Signaling, 12075S |

### Example 2: Solid-Phase Peptide Synthesis

Peptides were synthesized on Rink Amide MBHA resin via the standard Fmoc solid-phase peptide synthesis (SPPS) chemistry. For Fmoc deprotection, the resin was agitated with piperidine (20% v/v in DMF, 3×5 min) and washed with DMF 5 times. To couple amino acids to the resin, a DMF solution of Fmoc-AA (protection group)-OH (5 eq.), HBTU (4.75 eq), and DIEA (15 eq.) was mixed for 3 minutes, and then the mixture was added to the resin and agitated at room temperature for 1 hour. For peptide side-chain global deprotection and cleavage, the resin was washed with DCM and dried with compressed airflow. The beads were then cleaved in a TFA cleavage solution (TFA:TIPS:ddH2O; 95:2.5:2.5) for 2 hour. All peptides were purified on the RP-HPLC (DIONEX Ultimate 3000; Thermo Scientific, Germany) using a C18 reversed-phase preparative column (Kinetex. 5 μm EVO, 250 Å˜21.2 mm) or a C18 reversed-phase semi-preparative column (Kinetex. 5 μm EVO, 250 Å˜10 mm). The product was validated using MALDI-TOF MS (AB SCIEX TOF/TOF 5800, Framingham, MA) and lyophilized for long-term storage at −20° C.

### Example 3: Synthesis of Azide-Attached Poly-Lysine Dendrimers

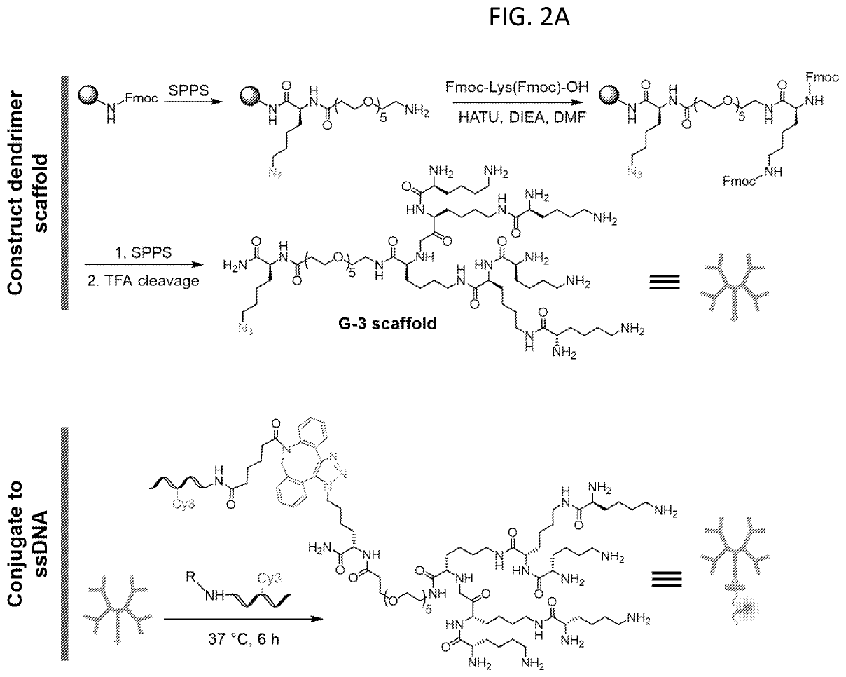

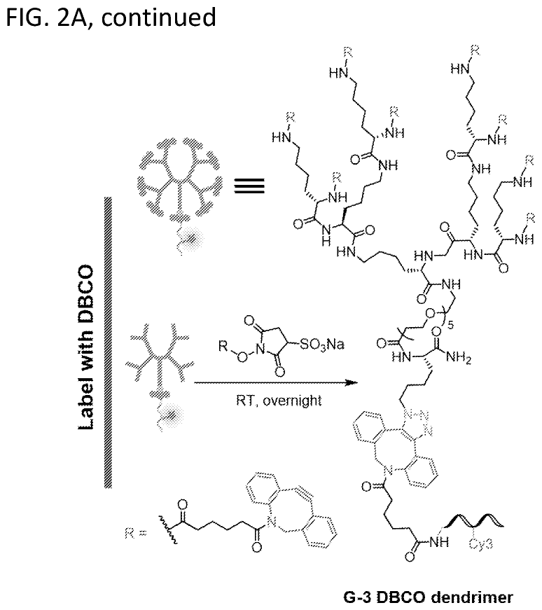

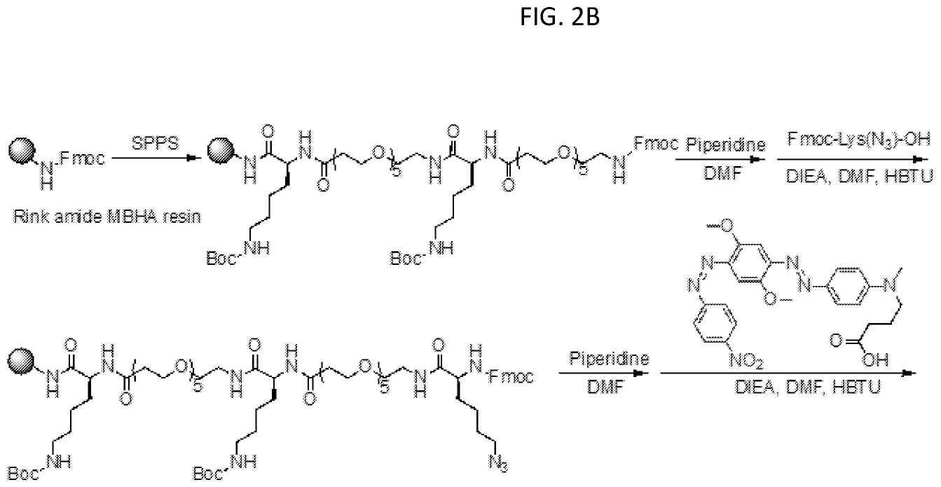

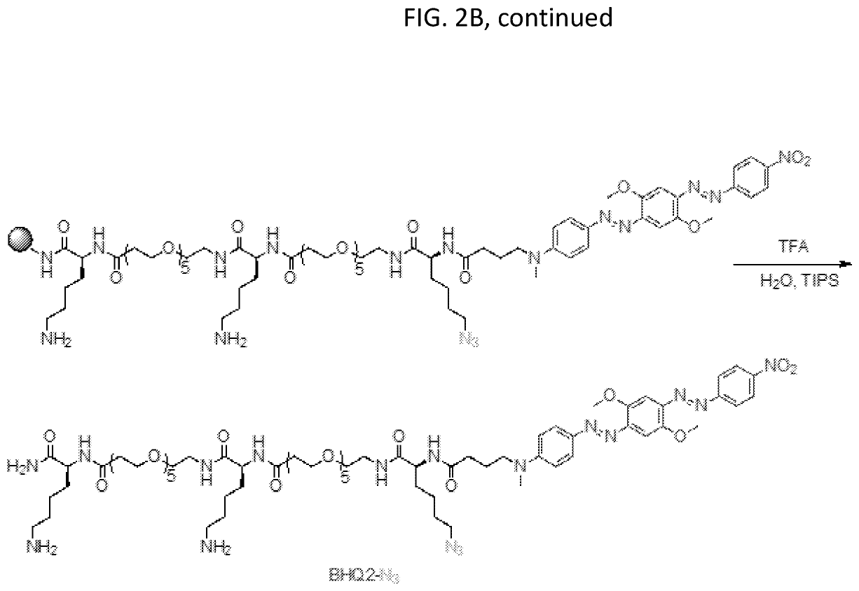

The azide-attached poly-lysine dendrimer scaffolds were synthesized on rink amide MBHA resin following standard SPPS protocols. The synthetic scheme is shown in FIG. 2A. Fmoc-l-Lys(N₃)—OH and Fmoc-PEG5-OH were attached to the resin first, followed by 3 or 4 rounds of Fmoc-1-Lys (Fmoc)-OH coupling. Before the cleavage, the Fmoc protecting groups were removed by piperidine. After the cleavage and HPLC purification, the dendrimer scaffolds were characterized by MALDI-TOF MS and lyophilized for storage.

G-3 poly-lysine dendrimer: 1H NMR (400 MHZ, D2O, as a TFA salt) δ 4.19 (t, J=7.1 Hz, 1H), 4.16-4.04 (m, 3H), 3.90 (t, J=6.6 Hz, 2H), 3.80 (t, J=6.6 Hz, 2H), 3.66 (t, J=5.9 Hz, 2H), 3.63-3.51 (m, 16H), 3.48 (t, J=5.3 Hz, 2H), 3.32 (dt, J=10.6, 5.3 Hz, 1H), 3.27-3.17 (m, 3H), 3.16-2.96 (m, 8H), 2.87 (dd, J=14.9, 7.3 Hz, 8H), 2.46 (t, J=5.9 Hz, 2H), 1.89-1.70 (m, 9H), 1.70-1.53 (m, 16H), 1.52-1.37 (m, 9H), 1.36-1.17 (m, 17H), 1.14 (t, J=7.3 Hz, 3H). 13C NMR (100 MHz, D2O, as a TFA salt, the signal of TFA was not included) δ 177.01, 174.36, 173.68, 173.29, 173.13, 169.53, 169.48, 169.26, 69.56, 69.37, 68.71, 66.71, 54.25, 54.11, 53.76, 53.52, 53.10, 52.68, 50.82, 46.65, 39.29, 39.16, 38.94, 38.87, 35.75, 30.70, 30.43, 30.36, 28.03, 27.66, 27.94, 27.89, 27.47, 26.34, 22.52, 22.42, 21.32, 21.16, 8.20.

G-4 poly-lysine dendrimer: 1H NMR (400 MHZ, D2O, as a TFA salt) δ 4.26-3.99 (m, 8H), 3.90 (t, J=6.6 Hz, 4H), 3.80 (t, J=6.6 Hz, 4H), 3.66 (t, J=5.9 Hz, 2H), 3.61-3.49 (m, 16H), 3.48 (t, J=5.3 Hz, 2H), 3.30 (dd, J=12.4, 7.1 Hz, 1H), 3.26-3.16 (m, 3H), 3.16-2.93 (m, 16H), 2.87 (dd, J=15.0, 7.3 Hz, 16H), 2.46 (t, J=5.9 Hz, 2H), 2.01-1.66 (m, 18H), 1.69-1.45 (m, 32H), 1.49-1.05 (m, 50H). 13C NMR (100 MHz, D2O, as a TFA salt, the signal of TFA was not included) δ 176.99, 174.33, 173.71, 173.38, 173.13, 169.52, 169.28, 169.23, 69.57, 69.41, 68.73, 66.73, 54.25, 54.09, 53.75, 53.52, 53.10, 52.71, 50.82, 46.64, 39.30, 38.85, 35.76, 30.82, 30.67, 30.41, 30.37, 28.12, 28.04, 27.90, 27.47, 26.34, 26.29, 22.56, 22.43, 21.33, 21.19, 8.20.

### Example 4: Conjugation of DBCO to ssDNA-Cy3-NH₂

50 μL of ssDNA-Cy3-DBCO solution (250 μM, in 1×PBS, pH 7.4) was mixed with 10 μL of DBCO-Sulfo-NHS solution (12.5 μL, 10 mM in 1×PBS) for 2 hours. After incubation, the mixture was purified by Zeba spin columns to remove unreacted small molecules. The concentration of ssDNA-Cy3-DBCO was determined by UV-Vis absorbance at 550 nm. The quality of the reaction was confirmed by FPLC and HPLC.

### Example 5: Conjugation of the Poly-Lysine Dendrimers (1 and 2) to ssDNA

50 μL of ssDNA-Cy3-DBCO solution (200 μM, in 3×PBS, pH 7.4) was mixed with 10 μL of poly-lysine dendrimer scaffold (10 mM, in 3×PBS, pH 7.4) at 37° C. for 6 hours. After incubation, the mixture was purified by FPLC to obtain ssDNA-dendrimer scaffold conjugates. The concentrations of the conjugates were determined by their UV-Vis absorbance at 550 nm.

### Example 6: Labeling the ssDNA-Dendrimer Conjugates (3 and 4) with DBCO

50 μL of ssDNA-dendrimer scaffold conjugates (100 μM in 3×PBS) was mixed with 20 μL of DBCO-Sulfo-NHS ester solution (10 mM for 3rd generation, 20 mM for 4th generation, in 3×PBS, pH 7.4). The mixture was incubated at room temperature overnight. The final products were purified by FPLC and stored at 4° C. The concentrations of the DBCO dendrimers and the number of DBCO on each dendrimer were determined by UV-Vis absorbance at 550 nm and 310 nm, respectfully.

### Example 7: Immobilization of the Dendrimer on a Glass Surface

A 16-well (50 μL each) PDMS slab was placed onto a microscope glass slide patterned with ssDNA strands that are complementary to the oligonucleotide sequence on DBCO dendrimers. Each well was blocked with 50 μL of 1% BSA in 1×PBST at room temperature for 1 hour. Afterward, the blocking solution was removed, and 45 μL of dendrimer (100 nM in 1% BSA, 1×PBST) was added to each well. After 1 hour of incubation at room temperature, each well was washed with 1×PBST three times. At this point, the dendrimer was immobilized on the device and ready to use.

### Example 8: Generation of the FA-N3 Response Curve Via a Surface-Based Competition Assay

The 3rd or 4th generation dendrimer was immobilized on the glass slide surface (described above). To generate a working curve of FA-N3, 45 μL of FA-N3 (various concentrations in 1×PBST) was added to each well. The slide was incubated at 37° C. for 2 hours, allowing FA-N3 to react with the DBCO groups on the dendrimer. The FA-N3 solution was then removed, and each well was washed with 1×PBST three times. 45 μL of BHQ2-N3 solution (25 μM in 1×PBST) was added to each well, and the slide/PDMS device was incubated at 37° C. for 1 hour, allowing BHQ2-N₃ to react with unoccupied DBCO sites. After removing the BHQ2-N₃ solution, and the device was washed with 1×PBST three times. The PDMS slab was then removed, and the slide was washed with 1×PBS, 50% 1×PBS/water, and water. The slide was then air-dried, and the fluorescence intensities were obtained using a Genepix microarray scanner. Experiments were run in quadruplets to get the error bar.

### Example 9: BHQ2-N3 Optimization on the Surface Platform

The 3rd generation dendrimer was first immobilized on the glass slide as described above. To scan for an optimal BHQ2-N₃ concentration, 45 μL of FA-N₃ (100 μM in 1×PBST) was added to each well. The device was left at 37° C. for 2 hours. The FA-N₃ solution was taken out, and each well was washed with 1×PBST three times. 45 μL of BHQ2-N₃ (various concentrations in 1×PBST) was then added to each well, and the device was kept at 37° C. for 1 hour, allowing BHQ2-N₃ to react with unoccupied DBCO sites. Afterward, the solution was removed, and the device was washed with 1×PBST three times. After the 16-well PDMS slab was removed from the device, the glass chip was washed with 1×PBS, 50% 1×PBS/water, and water. The chip was air-dried, and the fluorescence intensities were obtained using a Genepix microarray scanner. Experiments were run in quadruplets to obtain the error bar.

### Example 10: Cell Culture

U87vIII cell line was provided as a gift by Prof. Paul Mischel (UCSD, San Diego, U.S.A.). Cells were cultured in Dulbecco's Modified Eagle Media with high glucose, pyruvate (DMEM, Thermo), heat-inactivated fetal bovine serum (FBS), 100 U/mL of penicillin-streptomycin, and 250 ng/ml Amphotericin B (Thermo) in a humidified 5% CO₂ (v/v) incubator at 37° C.

### Example 11: FA-N₃ Tolerance Experiment

A 96-well plate was seeded with U87 cells (10,000/well), and the cells were allowed to grow overnight. Then the FA-N3 solution was added (100 μL per well, various concentrations) to the cells and incubated for 24 hours. After the incubation, the cell media was removed, and a resazurin solution (10 g/mL, in cell media) was added to the plate and incubated for 2 hours. The fluorescence was read by a plate reader (Biotek, 540 nm excitation/590 nm emission).

### Example 12: FA-N₃ Uptake Experiment at the Bulk Level

The U87 cells were collected via centrifugation at 500 g for 5 min. The media was removed, and the cells were resuspended in FBS-free DMEM, resulting in a 4 M cells/mL suspension. 250 μL of the cell suspension was dispensed into Eppendorf tubes, and mixed with 250 μL of the FA-N3 solution (various concentrations, in FBS free DMEM). After 30 min incubation in the cell incubator, the cells were centrifuged at 500 g for 5 min and the resulting pellet was washed 3 times to remove any excess FA-N₃. Then, 100 μL cell lysis buffer was added to each tube, and kept on ice for 15 min. The tubes were then sonicated and vortexed for 5 min. Finally, the tubes were centrifuged at maximum speed for 10 min. The supernatant was collected and subjected to surface-based FA-N₃ detection.

### Example 13: Single-Cell Suspension Preparation

To harvest the cells, the culture media was gently aspirated without disturbing the cells. The cells were then washed with PBS and further treated with 0.05% trypsin for 5 min at 37° C., followed by the addition of an equal volume of culture media to terminate the trypsinization process. The collected cells were then pelleted via centrifugation at 500 g for 5 min. After discarding the supernatant, the cells were then disassociated as single cells and ready for tests.

For SCBC measurements, the as-prepared single cells were resuspended in warm FBS-free media supplemented with 10 mM FA-N₃ at 2 M cells/mL. After being incubated at 37° C. for 30 min and washed with FBS-free media 3 times, the collected cell pellet was resuspended in serum-free, biotin-free media. The concentration of the as-prepared single-cell suspension was 2 M cells/mL.

### Example 14: Single-Cell Metabolic/Proteomic Measurements

The SCBCs were fabricated according to well-established procedures (Xue et al (2015) J Am Chem Soc 137 (12): 4066-4069). DNA-encoded antibody library (DEAL) was grafted onto the surface through DNA hybridization to enable capture antibody arrays. Capture and detection antibodies employed are reported in Table 1. The FA surface probe was also incorporated onto the surface barcode through the same procedure.

The SCBC devices were operated following previously established protocols (Xue et al (2015) J Am Chem Soc 137 (12): 4066-4069)). Briefly, FA-N₃ detection was achieved by a surface-based competitive FRET assay, and protein detection was carried out by a sandwich immunofluorescence assay. The cells were loaded in the SCBC with FA-N₃ first. Then each cell was isolated and lysed on each chamber. Upon cell lysing, FA-N₃ and other analytes were released from the cell and captured on the surface. Subsequently, the detection cocktail containing BHQ2-N₃ and antibodies were added to the chamber, and the fluorescence readout was recorded.

### Example 15: Image Acquisition and Data Extraction

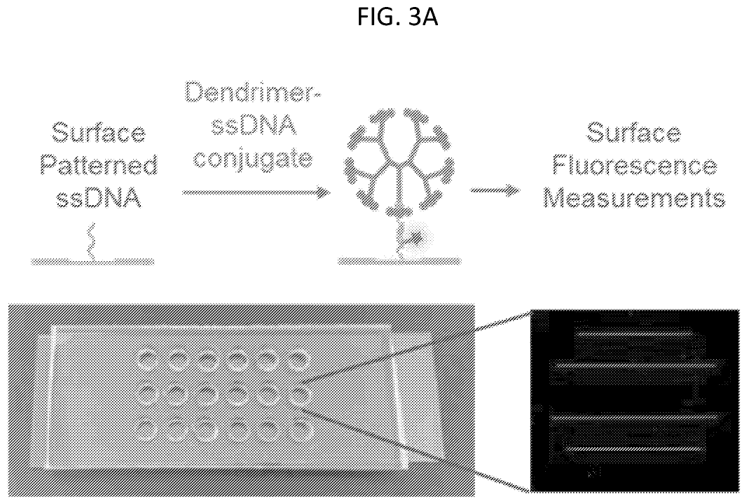

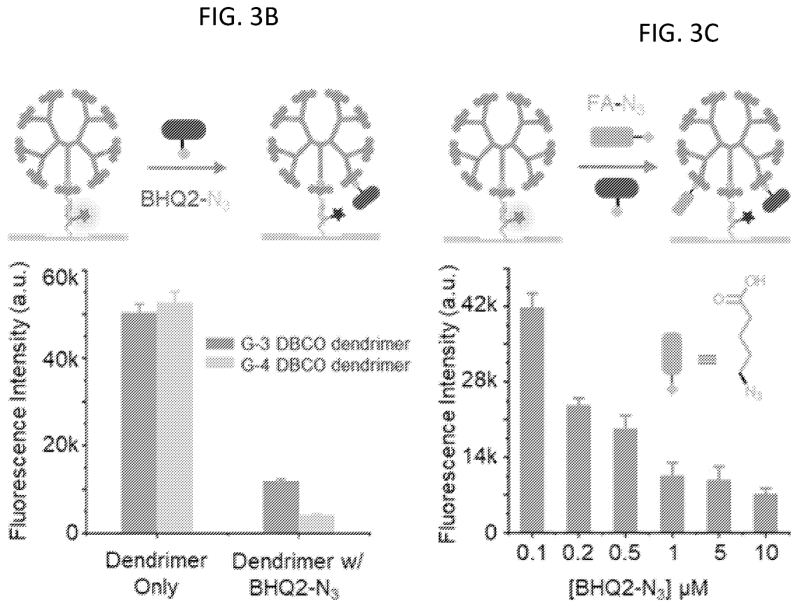

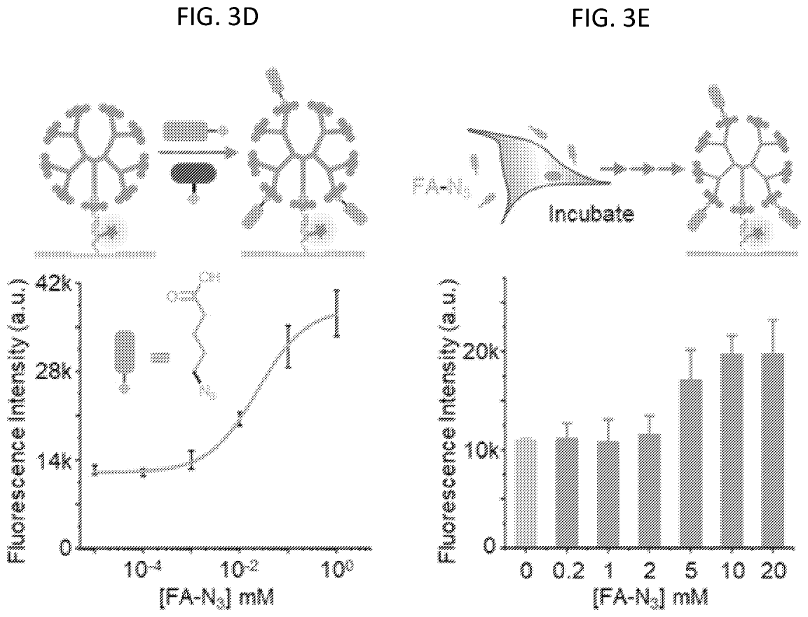

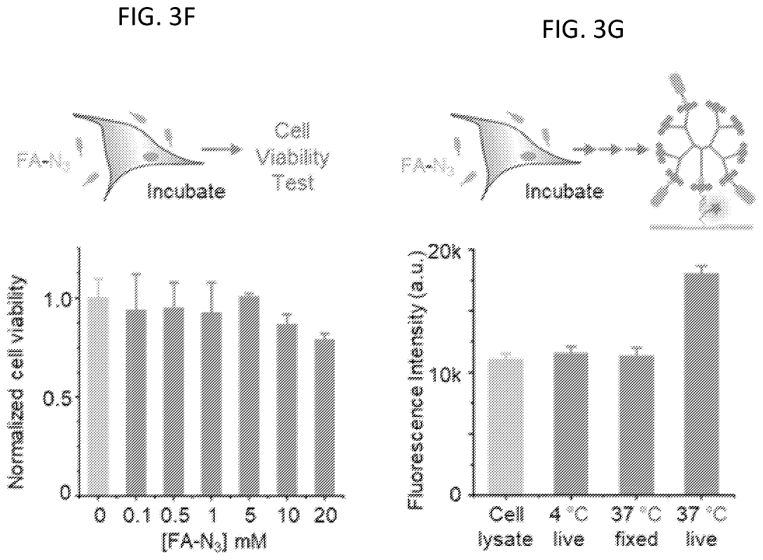

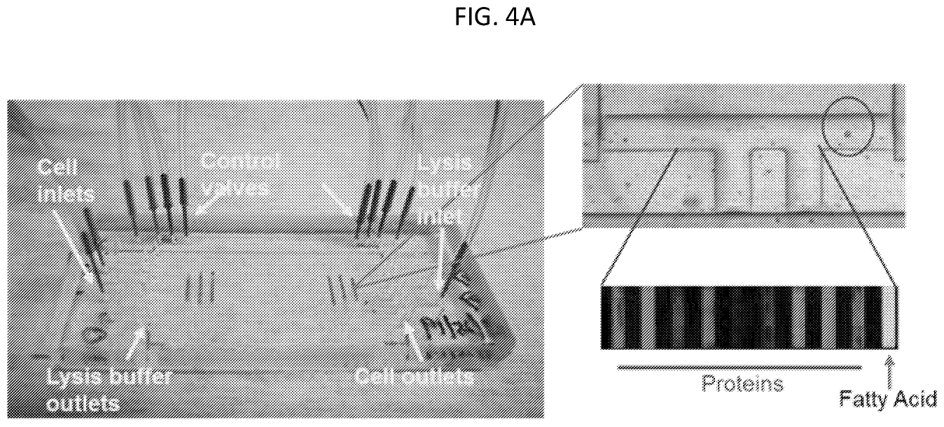

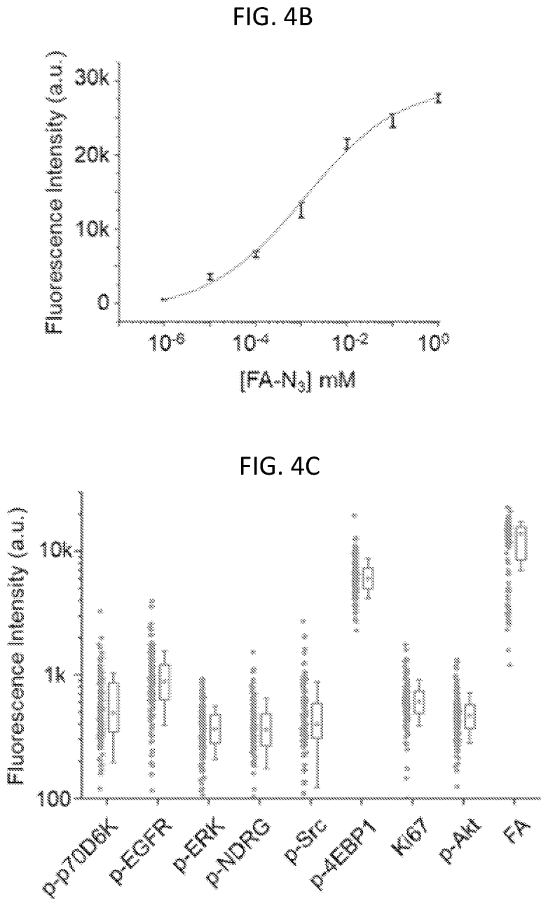

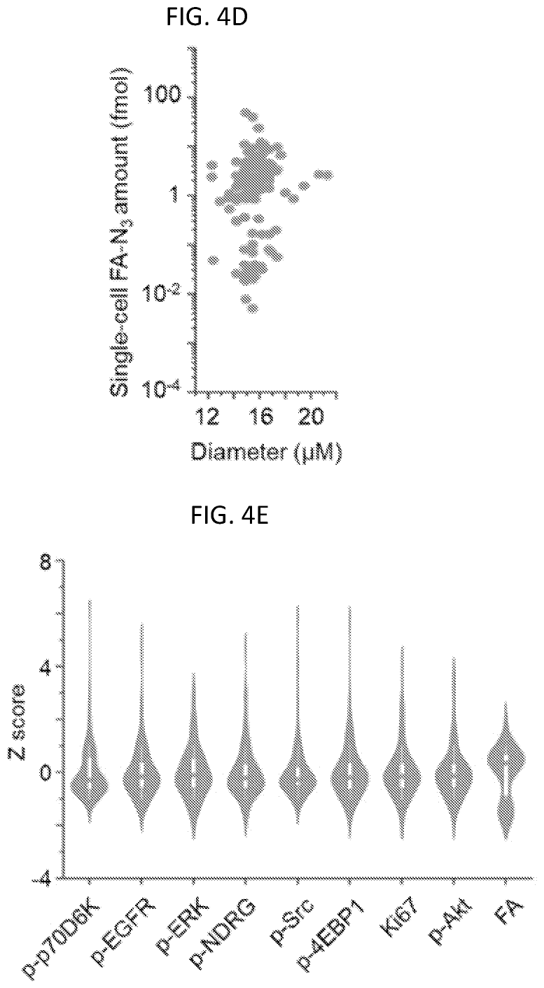

At the end of the mini well assay and the SCBC assay, the glass slides were detached from the PDMS device, and the surface fluorescence was analyzed using a microarray scanner (Genepix 4400A, 532 nm excitation/standard green filter; 635 nm excitation/standard red filter). Examples of the raw images are shown in FIG. 3A and FIG. 4A. To extract the quantitative data, the fluorescence intensity at each pixel was exported using the Genepix software. The median fluorescence intensity value of the pixels on each barcode strip was used as a data point for that specific analyte.

### Example 16: Statistical Analysis

The single-cell data was obtained as a matrix, where each row represented a single cell, and each column was the intensity of one analyte. Statistical analysis of this dataset was performed in OriginPro 2019b® software. The dataset was first standardized on each column to obtain the Z score of each value. The Euclidean distance values between data points were calculated and tabulated as a matrix. This distance matrix was then used as the input for agglomerative hierarchical clustering using Ward's method. To perform principal component analysis, the standardized single-cell dataset was used as input. The correlation coefficient was directly calculated from the data set by Spearman's rank method. Analyte-analyte correlation networks were generated by running the calculation through all the analyte pairs in the panel, and only those significant correlations (with Bonferroni correction) were shown in the networks. The network was presented as a Circos plot (Krzywinski et al (2009) Genome Res 19 (9): 1639-1645).

### Example 17: Drug Synergy Measurements

To ensure optimal cell attachment, 100 μL of 10 μg/mL laminin was added into each well of a 96-well plate, and the plate was incubated at 4° C. overnight. Afterward, the plate was washed with PBS three times. 100 μL of U87vIII cell suspension at 50 k/mL was then added into each well, and the plate was incubated at 37° C. overnight. On the second day, the media was changed to 200 μL of fresh media containing various concentrations of trimetazidine, and LY2584702. The cells were cultured for another 48 hrs. Subsequently, 20 μL of 0.2 mg/mL resazurin PBS solution was added into each well, followed by incubation at 37° C. for 3 hrs. The resulting fluorescent signals were recorded by a plate reader (560 nm excitation/590 nm emission).

The synergy scores of the two drugs were calculated by using the following equation:

$$SA , B = IA , B - ( IA + IB - IA \times IB )$$

where SA, B is the synergy effect between drugs A and B; IA,B is the cell-killing efficiency by using the combination of drug A and B; while IA and IB are the cell-killing efficiencies from independent doses of drug A or B, respectively.

### Example 18: Results and Discussion

As described above, we chose azide-modified fatty acids (FA-N₃) as surrogates and demonstrate a surface-based chemical method for detecting fatty acid uptake. The azide group is chemically inert, and its small size promises a better mimicry to natural fatty acids. We further adapted this method to the SCBC platform to achieve the multiplex analysis of fatty acid uptake with critical signaling proteins from the same single cancer cells.

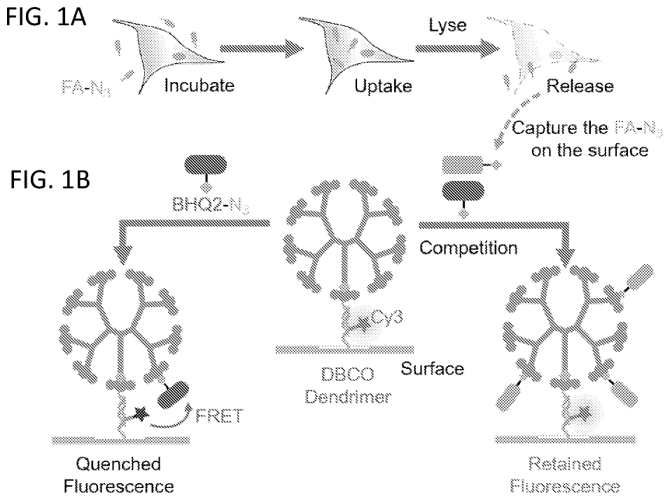

The fatty acid uptake analysis design is shown in FIG. 1A and FIG. 1B. Cells take up the FA-N₃ and then release them upon lysis. The released analogs can then be quantified through a surface-based competition assay. A straightforward method for detecting these azide-bearing analogs is to use the azide-alkyne click chemistry. Because of the aim to multiplex the fatty acid uptake analysis with protein profiling, Cu cannot be used to catalyze the click reaction due to its incompatibility with the immunofluorescence-based SCBC platform. Consequently, Cu-free click chemistry, e.g., strain-promoted azide-alkyne cycloaddition (SPAAC) must be used. However, azide-based SPAAC chemistry is limited by its low reaction rate. Even with the best reactant-dibenzocyclooctyne (DBCO), the rate constant is only 0.31 M⁻¹ S⁻¹ (Debets et al (2010) Chemical Communications 46 (1): 97-99). Such a reaction rate is not suitable for detecting the low amount of fatty acids in single cells. For instance, 50 μM azide-fatty acid and 10 μM DBCO-COOH lead to negligible changes after 3 hours of incubation (data not shown).

In order to improve the SPAAC reaction rate, we proposed to increase the local DBCO concentration. We hypothesized that a dendritic structure terminated with densely packed DBCO groups could effectively capture the azide-modified fatty acids (FIG. 1B). Nevertheless, this prominent hydrophobicity also makes it challenging to implement this detection in an aqueous detection environment.

To overcome the hydrophobicity problem, we proposed to conjugate the dendrimer to a single-strand DNA oligomer (ssDNA). This conjugation also helps to make the SPAAC-based detection compatible with the SCBC platform. We first synthesized G-3 and G-4 dendrimer scaffolds using lysine as building blocks and appended azido-lysine polyethylene glycol [Lys(N₃)-PEG] linkers (FIG. 2A); and HPLC of the synthesized G-3 polylysine dendrimer scaffold showed desired product. HPLC was performed on a Thermo Ultimate 3000BX instrument, using a C18 reversed-phase column (Phenomenex, Kinetex 5 μm EVO, 250×21.2 mm). The solvents used were A: H2O with 0.1% TFA, B: acetonitrile with 0.1% TFA. The method used was 0-75% B in 30 min, linear gradient. Mass spectrometry of the 3rd generation (G-3) polylysine dendrimer scaffold (MALDI-TOF, AB Sciex 5800) showed desired product: [M+H]+ calculated: 1359.94, found 1360.21). 1H-NMR spectrum of the G-3 polylysine dendrimer scaffold showed desired product. HPLC of the synthesized G-4 polylysine dendrimer scaffold showed desired product. HPLC was performed on a Thermo Ultimate 3000BX instrument, using a C18 reversed-phase column (Phenomenex, Kinetex 5 μm EVO, 250×21.2 mm). The solvents used were A: H2O with 0.1% TFA, B: acetonitrile with 0.1% TFA. The method used was 0-75% B in 30 min, linear gradient. Mass spectrometry of the 4th generation (G-4) polylysine dendrimer scaffold (MALDI-TOF, AB Sciex 5800) showed desired product: [M+H]+ calculated: 2384.70, found 2385.07). 1H-NMR spectrum of the G-4 polylysine dendrimer scaffold showed desired product. We then conjugated the dendrimers to a ssDNA oligomer through a SPAAC reaction (FIG. 2A); and FPLC and HPLC chromatograms of the synthesized ssDNA-Cy3-DBCO showed the correct product. The FPLC was performed on a Cytiva AKTA Pure instrument with a size-exclusion chromatography column (Cytiva, Superdex 30 increase 10/300 GL). An NH₄HCO₃ solution (50 mM in water, pH 7.8) was used as the eluent. The peak absorbance at 310 nm was 0.11, which corresponded to 1 DBCO group per DNA. The HPLC was performed on a Thermo Ultimate 3000 instrument, using a C18 reversed-phase column (Phenomenex, Kinetex 2.6 μm EVO, 250×4.6 mm). The solvents used were A: 400 mM HFIP (1,1,1,3,3,3-Hexafluoro-2-propanol)/2.3 mM triethylamine in water, pH 7.0, B: methanol. The method used was 0-100% B in 30 min, linear gradient. FPLC chromatography of the synthesized ssDNA-dendrimer (G-3 polylysine and G-4 polylysine) scaffolds showed the desired products. The FPLC was performed on a Cytiva AKTA Pure instrument with a size-exclusion chromatography column (Cytiva, Superdex 30 increase 10/300 GL). 3×PBS solution (pH 7.5) was used as the eluent. Note that high salt condition was critical to solubilizing the product. Lower salt concentrations led to rapid precipitation. Lastly, we installed DBCO groups on the dendrimer and obtained the dendrimer-DNA conjugate (FIG. 2A); and FPLC chromatograms of the final ssDNA-dendrimers (G-3 DBCO and G-4 DBCO) showed desired products. The FPLC was performed on a Cytiva AKTA Pure instrument with a size-exclusion chromatography column (Cytiva, Superdex 30 increase 10/300 GL). An NH₄HCO₃ solution (50 mM in water, pH 7.8) was used as the eluent. For G-3 DBCO, the peak absorbance at 310 nm was 0.89, which corresponded to 8 DBCO groups per DNA. For G-4 DBCO, the peak absorbance at 310 nm was 1.84, which corresponded to 16 DBCO groups per DNA. It is worth pointing out that it was important not to introduce DBCO groups until the very last step, or the strong hydrophobicity of DBCO groups would prevent the subsequent DNA conjugation (data not shown).

In our detection scheme (FIG. 1A and FIG. 1B), the dendrimer-ssDNA conjugate has an embedded Cy3 moiety that provides a fluorescence signal. This signal can be quenched later by a BHQ2-N₃ molecule through the Förster resonance energy transfer (FRET) mechanism. This BHQ2-N₃ molecule can be synthesized through the SPPS process (FIG. 2B. Mass spectrum BHQ2-N3 (MALDI-TOF) showed desired product: [M+H]+ calculated: 1498.82, found 1498.83). The 1199 peak corresponds to the photolytic cleavage of the azo bond (Wyplosz, N. Laser desorption mass spectrometric studies of artists' organic pigments. University of Amsterdam, 2003). In the presence of a large amount of FA-N₃ molecules, the DBCO sites are occupied and the chance of having a BHQ2-N₃ molecule on the dendrimer decreases. Therefore, FA-N₃ molecules can help retain the Cy3 fluorescence. The more FA-N₃ molecules there are, the fewer DBCO sites remain available for BHQ2-N₃ attachment. In this manner, the amount of FA-N₃ correlates with the retained fluorescence.

To validate our design, we prepared a mini well surface assay device (FIG. 3A). This device has two parts: a poly-dimethylsiloxane (PDMS) elastomer slab with mini wells and a glass slide with ssDNA patterned on the surface. Because of the complementary sequences, this surface-patterned ssDNA enables the immobilization of the DBCO-ssDNA conjugate. With this fully assembled assay device, we could test our proposed reactions by assessing the Cy3 fluorescence on the surface.

We first tested if the DBCO dendrimer could successfully capture the BHQ2-N₃ molecule. As shown in FIG. 3B, the surface fluorescence signals decreased significantly after BHQ2-N₃ incubation, which proved that BHQ2-N₃ successfully reacted with the DBCO moieties on the dendrimer and quenched the Cy3 fluorescence. This result supported our hypothesis that the dendrimer structure could increase the local DBCO concentration and capture azide-bearing molecules efficiently. It is also worth noting that the G-4 dendrimer led to a slightly stronger quenching result, which was expected due to its higher DBCO concentration. Nevertheless, considering that the G-4 dendrimer was more difficult to synthesize than the G-3 one and that the difference in the quenching efficiency was not prominent, we decided to use the G-3 dendrimer for the subsequent studies. To prove that the dendrimer structure was necessary, we also performed a similar experiment using a Cy3-ssDNA presenting only one DBCO group. In this case, the fluorescence was not quenched (data not shown).

Because FA-N₃ and BHQ2-N₃ are expected to compete for the DBCO sites, the resulted fluorescence readout in response to the FA-N₃ amount should be adjustable by changing the concentrations of BHQ-N₃. To identify a potentially optimal quencher concentration, we evaluated how different concentrations of BHQ-N₃ compete with 100 μM of FA-N₃ (azidopentanoic acid, FIG. 3C). We chose 0.5 μM as the best BHQ-N₃ concentration because it led to significant, but not complete, quenching of the Cy3 signal, which would allow the detection of lower FA-N₃ concentrations. Using this quencher concentration, we generated a fluorescence response curve by varying the concentrations of the FA-N₃ (FIG. 3D). We confirmed that this method could detect FA-N₃ at the μM-level, which is expected to be suitable for single-cell analysis of fatty acid uptake.

We tested our detection scheme using U87VIII cells as our model system. These are human glioblastoma cells that exhibit constitutively amplified oncogenic signaling activities and harbor prominent metabolic plasticity. We first incubated these cells with varying concentrations of the azidopentanoic acid probe and confirmed that the probe was well-tolerated by the cells (FIG. 3E). To identify a suitable FA-N₃ concentration that would generate signals within the assay dynamic range, we used our dendrimer-DNA conjugate/BHQ2-N₃ system to evaluate the surrogate uptake at the bulk level. The results (FIG. 3F) showed that the cells took up the FA-N₃ probe in a concentration-dependent manner, and the signal leveled off above 10 mM FA-N₃. This saturation pattern supported the active transportation of the FA-N₃ probe, which was consistent with the pentanoic acid uptake mechanism. Taken together, these results indicated that incubating the cells with 5 mM FA-N₃ was optimal for this assay.

Because of their hydrophobic tails, fatty acids may insert into the cell membrane. This partition is not an active transportation process and may confound the analysis. On the other hand, it was expected that the inserted probe molecules would remain in the membrane fragments upon cell lysis and not be detected by the assay. To assess this potential interference, we incubated the cells with the FA-N₃ probe at 4° C. and used our platform to quantify the FA-N₃ in the cells. At such a low temperature, the cells would stop the active transportation of the probe, and any observed signal would originate from the nonspecific insertion into the cell membrane. As expected, the resulted signal was indistinguishable from the control (FIG. 3G). To rule out the possibility that the low-temperature treatment dampened the rate of the nonspecific insertion, we performed another experiment where we fixed the cells and then incubated them with the FA-N₃ probe at 37° C. Again, the resulted signal was negligible (FIG. 3G). Collectively, these results demonstrate that the assay was specifically reporting the FA-N₃ probes that were actively taken up by the cells.

We then moved on to incorporate the fatty acid uptake assay onto the SCBC platform (Xue et al (2015) J Am Chem Soc 137 (12): 4066-9). The SCBC has two parts, a two-layer PDMS microfluidic device and a glass slide with patterned ssDNA barcode stripes. The device has 416 programmable microchambers that can trap and segregate single cells. These chambers allow on-chip cell lysis, and they are coupled with the DNA barcode stripes that enable multiplex fluorescence measurements (FIG. 4A). Here, the dendrimer-ssDNA conjugate was introduced to the microfluidics chambers and immobilized onto the surface through DNA hybridization, similar to the mini well assay described above. Using the SCBC device, we first confirmed that the nonspecific interaction between the FA-N₃ probe and the PDMS device was negligible. Assessment of the nonspecific binding between azidopentanoic acid (FA-N₃) and the PDMS chip was carried out as follows. 1 mM of the FA-N₃ probe was flown through the PDMS device and the resulting solution concentration was compared with the 1 mM FA-N₃ probe solution. Quantitation was performed using NMR (400 MHZ, D₂O). DMSO was used as an internal standard to calculate the FA-N₃ probe concentration. Three data points were collected. No statistical difference was observed using t-test). We then generated a fluorescence response curve by varying the FA-N₃ concentration (FIG. 4B). The result demonstrated improved sensitivity compared with the mini well assay. The sensitivity in-crease was consistent without prior observations on the SCBC platform. The contributing factors include the high-efficiency flow-based washing that decreased the back-ground and the confined chambers that promoted surface binding. Using the 3SD/slope method, we estimated the limit of detection to be 2 nM.

The dendrimer-ssDNA conjugate can be mixed with a cocktail of antibody-ssDNA conjugates to enable multiplex quantitation. As a proof of concept, we quantified the azidopentanoic acid uptake, critical signaling protein levels, and cell proliferation marker (Ki67) in U87VIII single cells. As shown in FIG. 4C, fatty acid uptake abilities varied significantly among cells. Using the standard curve described above, we were able to calculate the fatty acid concentrations in the chambers, which ranged from high nM to low μM. Considering that the chamber volume was 2 nL, we calculated that each cell took up low fmol-level FA-N₃ molecules, reaching high μM to low mM intracellular concentration (data not shown). We further investigated if the FA-N₃ uptake was dictated by the cell volume. As shown in FIG. 4D, there was no correlation between them. This result was expected because we have proven before that our assay only detects the probe molecules resulted from active transportation, whose rate was not necessarily linked to the cell size.

In order to better compare the heterogeneity among analytes, we standardized the raw data (FIG. 4C) to generate a Z-score violin plot (FIG. 4E), which provided a direct visualization of the analyte distribution. We found that the fatty acid uptake heterogeneity was different from those associated with signaling proteins. Most notably, fatty acid uptake had no outliers (|Z|>2), while all the protein levels showed outliers, many of which even extended beyond Z=4. The distribution of fatty acid uptake also exhibited two subpopulations corresponding to low (Z<−1) and high (Z>0) uptake activities. Such a clear subpopulation separation was not observed in the protein levels. Moreover, this fatty acid up-take distribution was different from the glucose and glutamine uptake results in previous studies on the same cell line (Xue et al (2015) J Am Chem Soc 137 (12): 4066-9; and Xue et al (2016) J Am Chem Soc 138 (9): 3085-93).

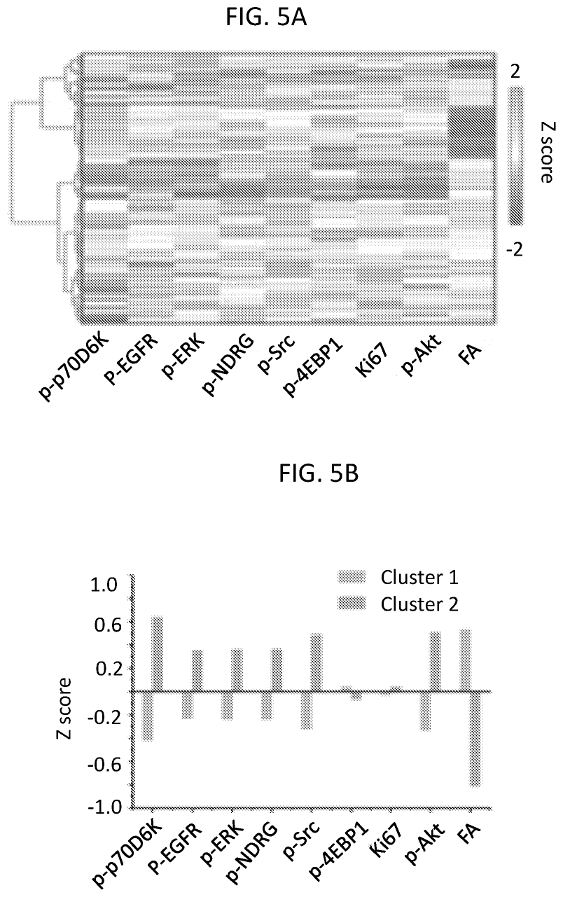

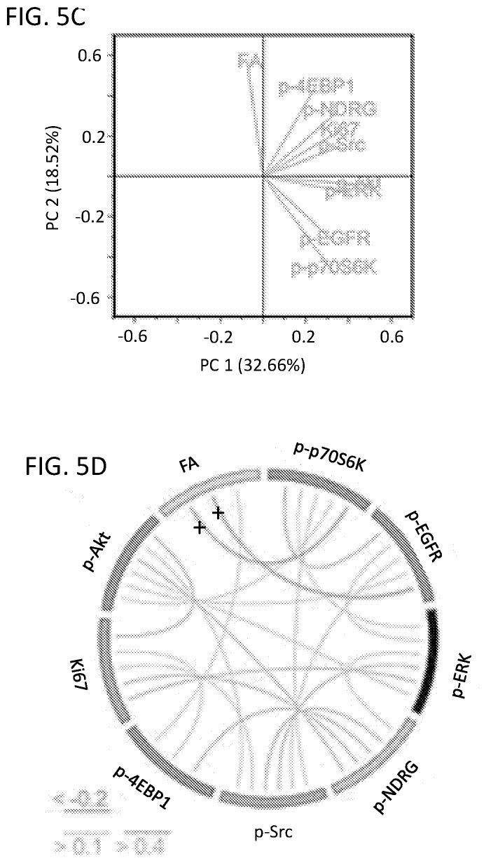

To further investigate the subpopulations, we performed agglomerative hierarchical clustering analysis (FIG. 5A). Based on the clusters' analyte levels (FIG. 5A and FIG. 5B), we found that cells with high fatty acid uptake exhibited low oncogenic signaling activities (p-EGFR, p-ERK, p-Src and p-Akt). This divergence pointed to a compensatory relationship between glycolysis and fatty acid metabolism. We also noticed that the levels of the cell proliferation marker, Ki67, did not vary between the two subpopulations. This result indicated that neither oncogenic signaling nor fatty acid metabolism was strongly associated with cell proliferation. Such an observation was consistent with our previous studies (Xue et al (2015) J Am Chem Soc 137(12):4066-9; and Xue et al (2016) J Am Chem Soc 138(9):3085-93).

In order to better evaluate how each analyte contributed to the global cellular heterogeneity, we performed principal component analysis (FIG. 5C). PC1 was primarily populated by oncogenic phosphoproteins, including p-Akt, p-ERK, p-Src, etc., while fatty acid uptake was aligned along PC2 with minimal contribution to PC1. This orthogonality between fatty acid uptake and critical oncogenic signaling matched with the clustering results. GBM is known to be a highly glycolytic malignancy driven by its commonly altered protein signaling in EGFR/PI3K/Akt and MAPK/ERK pathways (Pavlova et al. (2016) Cell Metabolism 23 (1): 27). Activation of EGFR and downstream PI3K/Akt signaling in many GBM cells directly drives cellular glucose uptake and glycolysis by enhancing both the transcriptional expression and translocation to the cell surface of glucose transporters (GLUTs) as well as activating several enzymes in the glycolytic pathway, including hexokinase and PKM2 (Wieman et al. (2007) Molecular Biology of the Cell 18 (4): 1437; Elstrom et al. (2004) Cancer Research, 64 (11): 3892; Liang et al. (2016) Nature Communications 7:12431). The MAPK/ERK signaling can also promote aerobic glycolysis via induction of transcriptional factor c-Myc (Papa et al. (2019) Oncogene 38 (13): 2223). GBM cells have been reported to be mostly relying on glycolysis as the primary source of ATP in standard culture conditions (Kant et al. (2020) Cell Death & Disease 11 (4): 253)). Therefore it was not surprising that fatty acid uptake was slightly decoupled from those oncogenic signaling under normal culture conditions when the primary energy source in GBM cells is glucose (Kant et al. (2020) Cell Death & Disease 11 (4): 253; Nagarajan et al. (2021) Cancer & Metabolism 9 (1): 2; and Caniglia et al. (2021) Theranostics 11 (5): 2048-2057). Moreover, we observed a strong divergence between fatty acid uptake and p-p70S6K as well as a corporative relationship between fatty acid uptake and p-4EBP1. Because p70S6K and 4EBP1 are two main effectors downstream of mTOR (Liu et al. (2020) Nature Reviews Molecular Cell Biology 21 (4): 183-203), these findings hinted that fatty acid uptake was regulated by mTOR signaling, possibly differentially controlled by the 4EBP1 and S6K axes. These results were consistent with the critical role of mTOR signaling in lipid homeostasis and fatty acid metabolism (Tsai et al. (2016) Cell Reports 16 (7): 1903-1914; Koundouros et al. (2020) British Journal of Cancer 122 (1): 4-22; and Ricoult et al. (2013) EMBO Rep 14 (3): 242-251). Nevertheless, PC1 and PC2 only collectively captured half of the global cellular heterogeneity variance (data not shown). Therefore, the conclusions drawn from analyzing PC1 and PC2 required further support.

We then sought to scrutinize the interactions between analytes. We calculated the pairwise Spearman correlation values among all analytes, and the result is shown in FIG. 5D. We found strong correlations between the signaling proteins, which were indicative of highly coordinated oncogenic signaling network activities and consistent with previous studies on U87VIII cells. Notably, fatty acid showed negative correlations with p-p70S6K and p-EGFR. P70S6K is a downstream target of mTOR Complex 1 (mTORC1)—a critical regulator of cell proliferation and protein synthesis. Increasing evidence reveals that tumor cells can uncouple glycolysis from mitochondrial oxidation, allowing the use of additional fuel sources, such as amino acids and fatty acids, to meet their heightened metabolic needs (Pavlova et al. (2016) Cell Metabolism 23 (1): 27; Li et al. (2019) Nature Communications 10 (1): 3856; Anderson et al. (2018) Protein & Cell 9 (2): 216). GBM cells are also capable to utilize fatty acid metabolism to generate ATP to maintain survival under nutrient deprivation or therapeutic stress that limits their glucose consumption (Kant et al. (2020) Cell Death Dis 11 (4): 253). The anti-correlations between fatty acid uptake and EGFR/mTORC1 signaling echo the previously observed bioenergetic reprogramming towards fatty acid metabolism upon oncogene inhibition or cytostatic treatment in GBM (Caragher et al. (2020) Cancers 12 (11): 3126; Guo et al. (2009) Science Signaling 2 (101): ra82). Based on the strong anticorrelation between fatty acid uptake and p-p70S6K, we further hypothesized that co-inhibiting p70S6K and fatty acid metabolism would synergistically inhibit cell proliferation.

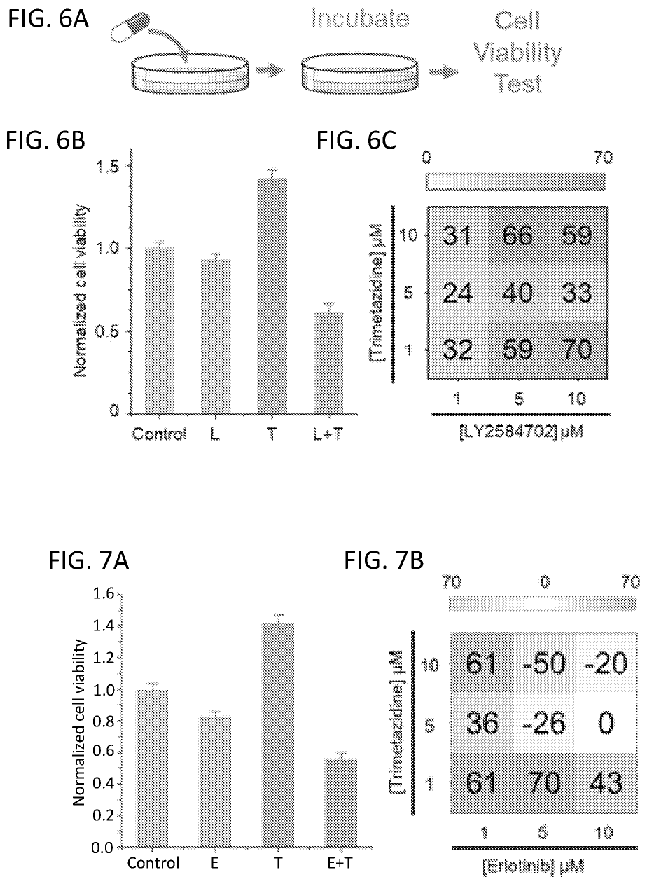

To test this hypothesis, we treated U87VIII cells with LY2584702 (a p70S6K inhibitor), trimetazidine (a fatty acid metabolism inhibitor), and a combination of them. It was evident that the combination synergistically inhibited cell proliferation (FIG. 6A). To quantitatively evaluate this synergy, we treated the cells with drugs under a concentration titration and calculated the synergy score using the BLISS definition of independence. As shown in FIG. 6B, we observed strong synergy between the two drugs across a wide range of concentrations. This result supported our hypothesis that p70S6K negatively regulated fatty acid metabolism in U87VIII cells. We further confirmed that such a synergistic therapy was also applicable to the parent EGFR wild-type U87 and a patient-derived GBM neurosphere GBM39 cells, suggesting that the observed anti-correlation between fatty acid uptake and mTORC1 may also exist in other GBM cells, independent of EGFR mutational status (data not shown).

Co-administering trimetazidine and erlotinib also resulted in improved inhibition of cell proliferation (FIG. 7A), with synergistic killing effects on U87VIII cells across different drug concentrations using a combination of 1 μM of trimetazidine and 1 μM of erlotinib (FIG. 7B).

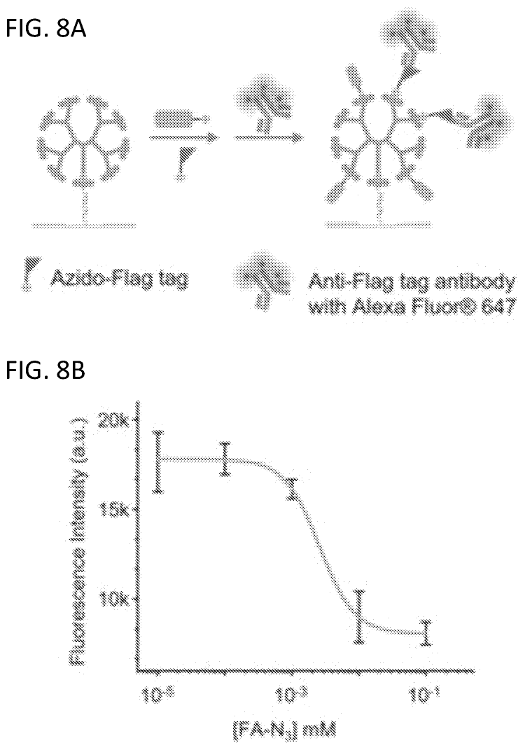

Lastly, we developed a detection strategy in addition to the competitive FRET for our dendrimer detection approach, namely, a competitive antibody detection system. As shown in FIG. 8A, azide-modified fatty acid analog was found to compete with azido-Flag (azide-DYKDDDDK; SEQ ID NO: 1) for the binding sites of the dendrimers. These Flag tags were readily detected by anti-Flag antibodies modified with AF647. FIG. 8B shows results of competitive binding with azido-Flag and detection with AF647. As can be seen, the surface fluorescence intensities negatively correlate with the FA-N3 concentrations.

In summary, we established a dendrimer-based detection scheme for profiling fatty acid uptake in single cells. Azide-modified analogs were used to probe the fatty acid influx, and surface-immobilized dendrimers with DBCO groups were used for detection. A competition between the fatty acid probes and an azide-modified detection reagent generated fluorescence signals in a concentration-dependent manner. This included BHQ2-azide quencher molecules paired with built-in fluorophore-modified dendrimer for competitive FRET detection, and azido-FLAG-tags with anti-FLAG-tag antibody for competitive antibody detection. By integrating this approach onto a microfluidics-based multiplex single-cell barcode chip platform, we were able to perform simultaneous analysis of fatty acid uptake and critical oncogenic signaling proteins in single cells. Moreover, the relationships between fatty acid influx, oncogenic signaling activities, and cell proliferation in single glioblastoma cells were resolved. It was found that p70S6K and 4EBP1 differentially correlated with fatty acid uptake. The approach demonstrated that co-targeting p70S6K and fatty acid metabolism synergistically inhibited cell proliferation. To our knowledge, this work provides the first example of studying fatty acid metabolism in the context of protein signaling at single-cell resolution and generated new insights into cancer biology. As such, the technology presented here can be extended to identify other potential regulatory mechanisms of fatty acid metabolism by including additional proteins into the panel, given that the appropriate antibody pairs are available. In addition, the dendrimer-based platform can be easily adapted to detect other azide-modified small molecules.

In view of the many possible examples to which the principles of the disclosure may be applied, it should be recognized that the illustrated examples should not be taken as limiting the scope of the invention. Rather, the scope of the invention is defined by the following claims. We therefore claim as our invention all that comes within the scope and spirit of these claims.

1. A surface comprising a dendrimer terminating in a plurality of densely packed azide-capturing groups, the dendrimer conjugated to a spacer comprising a single stranded nucleic acid hybridized to a complementary nucleic acid patterned on the surface in a spatially addressable array.

2. The surface of claim 1, wherein the spacer and the complementary nucleic acid comprise complementary barcode nucleic acids patterned on the surface in a spatially addressable barcoded array.

3. The surface of claim 2, wherein the surface is on a single-cell barcode chip.

4. The surface of claim 1, wherein the spacer is labeled with a reporter dye.

5. The surface of claim 1, further comprising a capture antibody reagent conjugated to a second spacer comprising a barcode patterned on the surface in a spatially addressable barcoded array.

6. The surface of claim 5, wherein the dendrimer and the capture antibody reagent are each individually spatially patterned in stripes in a microchamber of the single-cell barcode chip.

7. The surface of claim 1, wherein the azide-capturing groups are dibenzocyclooctyne (DBCO).

8. (canceled)

9. A cell-free composition comprising purified, monodisperse dendrimer terminated with a plurality of densely packed azide-capturing groups, the dendrimer conjugated to a spacer.

10. The composition of claim 9, wherein the spacer comprises a single stranded nucleic acid and/or a reporter dye.

11. The composition of claim 10, wherein the single stranded nucleic acid comprises a barcode nucleic acid.

12-14. (canceled)

15. The composition of claim 9, wherein the azide-capturing groups are dibenzocyclooctyne (DBCO).

16. (canceled)

17. A composition comprising an azide-modified quencher of a reporter dye, the azide-modified quencher comprising a hydrophilic polymer bearing an azide moiety and a quencher of the reporter dye.

18. The composition of claim 17, wherein the quencher of a reporter dye is a dark quencher.

19. The composition of claim 17, wherein the hydrophilic polymer comprises monomers selected from hydrophilic amino acid, polyethylene glycol, and combinations thereof and/or wherein the azide moiety is azido lysine.

20. (canceled)

21. The composition of claim 19, wherein the dark quencher is a Black Hole Quencher® selected from BHQ®-1, BHQ®-2, and BHQ®-3.

22. (canceled)

23. An assay comprising:
(a) contacting the surface of claim 1 with an azide-modified compound of interest and an azide-modified detection reagent under competitive binding conditions; and
(b) detecting the azide-modified detection reagent.

24. The assay of claim 23, wherein the azide-modified compound of interest is an azide-modified metabolite.

25-26. (canceled)

27. The assay of claim 23, wherein the spacer is labeled with a fluorophore and the azide-modified detection reagent comprises a quencher of the fluorophore, and wherein the detecting comprises measuring fluorescence resonance energy transfer (FRET).

28. The assay of claim 27, wherein the azide-modified quencher is selected from BHQ2-N3 and derivatives thereof.

29. The assay of claim 23, wherein the azide-modified detection reagent is an azide-flag tag.

30. The assay of claim 29, wherein the detecting of the azide-flag tag comprises contacting the surface with a fluorophore-labeled anti-flag tag antibody and measuring fluorescence thereof.

31. The assay of claim 23, wherein the surface further comprises a capture antibody reagent, and wherein the method further comprises:
contacting the surface with a protein sample and a detection antibody reagent capable of binding a protein of interest, and detecting binding of the detection antibody reagent to the protein of interest if present in the protein sample.

32. The assay of claim 31, wherein the detecting assesses the presence or absence of the protein of interest in the protein sample, and optionally, the level of the protein of interest in the protein sample or wherein the detection antibody reagent is a fluorophore-labeled anti-protein of interest antibody, and the detecting comprises measuring fluorescence thereof.

33-34. (canceled)

35. The assay of claim 31, wherein the azide-modified compound of interest is a metabolite, and wherein the metabolite and the protein of interest are detected in an isolated region on the same surface.

36. The assay of claim 35, wherein the isolated region on the same surface is a microchamber of a single-cell barcode chip.

37. (canceled)

38. The assay of claim 23, further comprising:
(i) contacting a cell with the azide-modified compound of interest;
(ii) lysing the cell so as to release the azide-modified compound of interest taken up by the cell in step (i),
wherein the contacting and lysing in steps (i) and (ii) is prior to contacting the surface in step (a).

39. The assay of claim 38, wherein the cell is a cancer cell or the cell is a single-cell in isolation.

40. (canceled)

41. A method of treating a cancer patient, the method comprising:
administering to a cancer patient in need thereof an effective amount of an inhibitor of fatty acid metabolism in combination with an effective amount of an inhibitor of a protein selected from p70S6K, pEGFR, or combinations thereof.

42. The method of claim 41, wherein the inhibitors of fatty acid metabolism, p70S6K, and pEGFR, are trimetazidine, LY2584702, and erlotinib, respectively.

43. (canceled)
